(19) United States
(12) Patent Application Publication
(10) Pub. No.: US 2022/0143552 A1
(43) Pub. Date: May 12, 2022

(54) FILTRATION

(71) Applicant: NVIGOREA AB, UPPSALA (SE)

(72) Inventor: ALBERT MIHRANYAN, Uppsala (SE)

(73) Assignee: NVIGOREA AB, UPPSALA (SE)

(21) Appl. No.: 17/598,546

(22) PCT Filed: Mar. 30, 2020
(86) PCT No.: PCT/EP20/58995

§ 371 Date: Sep. 27, 2021

(30) Foreign Application Priority Data
Mar. 29, 2019 (GB) 1904460.1

(51) Int. Cl. B01D 61/02; B01D 71/10; B01D 61/04; B01D 69/02; B01D 61/14; B01D 61/58

(52) U.S. Cl. CPC B01D 61/022; B01D 71/10; B01D 61/04; B01D 2325/04; B01D 61/147; B01D 61/58; B01D 2325/02; B01D 69/02

(57) ABSTRACT

The present invention provides method of removing particles from a feed fluid, the method comprising: passing the fluid through a first filtration medium having a thickness of from 5 to 20 μm, wherein passing the feed fluid through the first filtration medium provides a particle removal probability log10 reduction value (LRV) of greater than or equal to 1 for particles having a diameter of from about 10 to about 40 nm and a particle removal probability log10 reduction value (LRV) of greater than or equal to 3 for particles greater than about 40 nm in diameter; and passing the fluid through a second filtration medium having a thickness of from 20 to 70 μm (e.g. 20 to 45 μm) 20 to 45 pm, wherein passing the feed fluid through the second filtration medium provides a particle removal probability log10 reduction value (LRV) of greater than or equal to 3 for particles having a diameter of from about 10 to about 40 nm and a particle removal probability log10 reduction value (LRV) of greater than or equal to 3 for particles having a diameter of greater than or equal to about 40 nm; so as to retain at least a portion of the particles on each medium to produce a filtrate containing a lower concentration of the particles than the feed fluid.

The present invention relates to a method for removing particles from a feed fluid via the use of multiple filtration media, a kit-of-parts comprising multiple filtration media and the use of such a kit-of-parts in removing particles from a feed fluid.

Proteins feature various unique bioactive functions making them valuable therapeutic and nutraceutical commodities. However, all protein-based products derived from tissue cultures, human plasma, animals or plants possess the risk of viral contamination, which can be endogenous or adventitious. Therefore, ensuring the purity, and in turn the safety, of protein based therapeutic products is a prerequisite for their marketing. In particular, virus removal filtration is an established and critical process during the manufacture of protein-based drugs. In some instances, virus removal filtration is the single most expensive unit operation for entire bioprocessing.

Viruses can be removed from fluids by several means, including filtration (such as depth filtration or surface screening), partitioning and fractionation (such as centrifugation), and chromatography (such as ion-exchange, affinity or gel-permeation chromatography). Alternatively, viruses within fluids can be inactivated by chemical means (such as chaotropic agents, low pH environments, solvents and detergents) or physical means (such as the provision of heat and/or irradiation). However, the use of physical means in particular is often associated with undesired damage to other components in the fluid. Furthermore, some viruses are hard to inactivate by chemical means and become resistant to deactivation, e.g. reovirus or SV40. Especially challenging are small size non-enveloped viruses, such as parvoviruses. Additionally, even if the virus is chemically deactivated the virus markers still remain in the solution, which makes the quality assurance and validation of the deactivation process troublesome. Accordingly, filtration and, especially, size-exclusion filtration is the preferred mode of removing a virus from a fluid because it is both non-destructive, i.e. does not compromise the integrity of the biological sample of interest, and non-interfering, i.e. does not cause immune reactions.

Most viruses have a particle size in the range between 18 and 300 nm. For convenience, viruses are typically divided into two groups: large size viruses (those having diameters above 40 nm) and small size viruses (those having diameters in the range of from 18 to 40 nm). Accordingly, most proteins typically have a particle size below 18 nm, i.e. smaller than the smallest viruses, although some very large proteins also exist. From the industrial point of view, a large number of useful proteins (both recombinant and plasma-derived) feature particle sizes nm.

Virus removal filters normally feature very narrow and well-defined pore size distributions, which allows them to reject all types of viruses and other microorganisms, while ensuring passage of the proteins more or less unhindered. It is technologically difficult to control the pore size distribution in nm-range. Furthermore, virus removal filters require extensive integrity testing and validations and, therefore, their price is very high, with current listings up to 8,000 USD/m². Taking into account the cost of manufacturing protein-based drugs, the price of virus removal filtration is currently about 30,000-40,000 USD/kg recombinant protein. In this context, the virus removal filters used in bioprocessing are single-use disposable products.

One of the major drawbacks of using size-exclusion filters is that they are prone to fouling during operation, which can result in a loss of product recovery and/or the need for routine replacement of the filters.

The behaviour of size-exclusion filters with regard to fouling is difficult to predict because processing parameters such as pH and ionic strength of buffer; protein character and concentration; and applied trans-membrane pressure may greatly influence the yield of the product.

The fouling of size-exclusion filters greatly reduces the operational life-time of the filters and may stipulate filter oversizing to cope with the bioprocess. The latter results in increased manufacturing costs.

In an attempt to reduce the impact of fouling, current filtration processes typically include, sometimes multiple, pre-filtration steps using membranes with 0.1 to 0.2 μm pore sizes in order to remove undesirable high molecular weight protein impurities, unfolded proteins, and/or protein aggregates. However, a drawback of using such a pre-filtration step is that sub-0.1 μm impurities may still contaminate the product in low quantities and cause filter fouling. In the literature, these sub-0.1 μm impurities are sometimes referred to as soluble aggregates as opposed to insoluble protein aggregates retained on sterilizing grade 0.1 to 0.2 μm membranes. It is generally conceived that the removal of low quantities of soluble aggregates constitutes one of the greatest challenges during bioprocessing.

Alternatively, in order to remove soluble aggregates, pre-filtration steps using adsorptive type depth filters are also commonly utilised. Such prefilters may be composed of cellulose fibers with diatomaceous earth. It is well documented in the literature that these pre-filters increase the product yield during virus removal filtration. However, because they operate on adsorptive principles, these depth filters can be sensitive to pH and buffer strength changes. They are further dependent on contact time and they are expensive.

The prior art covers the use of adsorptive depth filters as well as charged or surface modified microfiltration (MF) membranes to remove high molecular weight impurities and/or aggregates from protein solutions in order to enhance the performance of virus filters.

However, despite the use of pre-filtration steps, filter fouling due to low quantities of soluble aggregates, high molecular weight protein impurities, or unfolded proteins is still a major problem which results in increased costs, increased material use and lower product yield.

It should also be mentioned that these undesired contaminants may possess immunogenic properties and thereby cause unwanted adverse clinical reactions.

Therefore, there is a need for an improved method for filtering a feed fluid, which eliminates or significantly reduces the quantities of soluble protein aggregates, high molecular weight impurities, and unfolded proteins.

The inventors have surprisingly found that by first passing a fluid through a first filtration medium having a thickness of from 5 to 20 μm, which provides a particle removal probability log10 reduction value (LRV) of greater than or equal to 1 for particles having a diameter of below or equal to about 40 nm and a particle removal probability log10 reduction value (LRV) of greater than or equal to 3 for particles greater than, or equal to, about 40 nm in diameter; the fouling of a finer second filtration medium, such as one having a thickness of from 20 to 70 μm (e.g. 20 to 45 μm), which provides a particle removal probability log10 reduction value (LRV) of greater than or equal to 3 for particles having a diameter of from about 10 to about 40 nm and a particle removal probability log reduction value (LRV) of greater than, or equal to, 3 for particles having a diameter of greater than, or equal to, about 40 nm, is reduced beyond that which would be expected and the lifetime of the second filtration medium is extended.

The inventors have also surprisingly found that by first passing a fluid through a first filtration medium having a thickness of from 5 to 20 μm and a pore size distribution such that the modal pore diameter is in the range of from 10 to 25 nm, the fouling of a finer second filtration medium, such as one having a thickness of from 20 to 70 μm (e.g. 20 to 45 μm) and a pore size distribution such that the modal pore diameter is in the range of from 10 to 25 nm, is reduced beyond that which would be expected and the lifetime of the second filtration medium is extended. While it is not entirely clear how this surprising effect is possible, the inventors speculate the importance of particle retention in the depth of the filter due to enhanced tortuosity of the pore network in the second filtration medium as compared to the first one.

### DETAILED DESCRIPTION OF THE INVENTION

According to the first aspect of the invention, there is provided a method of removing particles from a feed fluid, the method comprising:

passing the fluid through a first filtration medium having a thickness of from 5 to 20 μm, wherein passing the feed fluid through the first filtration medium provides a particle removal probability log10 reduction value (LRV) of greater than or equal to 1 for particles having a diameter of below or equal to about 40 nm and a particle removal probability log reduction log10 reduction value (LRV) of greater than or equal to 3 for particles greater than, or equal to, about 40 nm in diameter; and passing the fluid through a second filtration medium having a thickness of from 20 to 70 μm (e.g. 20 to 45 μm), wherein passing the feed fluid through the second filtration medium provides a particle removal probability log10 reduction value (LRV) of greater than or equal to 3 for particles having a diameter of from about 10 to about 40 nm and a particle removal probability log10 reduction value (LRV) of greater than, or equal to, 3 for particles having a diameter of greater than, or equal to, about 40 nm; so as to retain at least a portion of the particles on each media to produce a filtrate containing a lower concentration of the particles than the feed fluid.

In an alternative aspect, the invention provides a method of removing particles from a feed fluid, the method comprising:

passing the fluid through a first filtration medium having a thickness of from 5 to 20 μm, wherein passing the feed fluid through the first filtration medium provides a particle removal probability log10 reduction value (LRV) of greater than or equal to 1 for particles having a diameter of below or equal to about 40 nm and a particle removal probability log reduction log10 reduction value (LRV) of greater than or equal to 3 for particles greater than, or equal to, about 40 nm in diameter; and passing the fluid through a second filtration medium, wherein passing the feed fluid through the second filtration medium provides a particle removal probability log10 reduction value (LRV) of greater than or equal to 3 for particles having a diameter of from about 10 to about 40 nm and a particle removal probability log10 reduction value (LRV) of greater than, or equal to, 3 for particles having a diameter of greater than, or equal to, about 40 nm; so as to retain at least a portion of the particles on each media to produce a filtrate containing a lower concentration of the particles than the feed fluid. In this aspect, the second filtration medium may comprise porous materials such as synthetic or semi-synthetic polymers (for example polyvinylidene difluoride (PVDF), cuprammonium-regenerated cellulose, cellulose acetate, polyethylenesulfone (PES), polycarbonate) and porous ceramic materials.

Preferably, the first filtration medium has a pore size distribution such that the modal pore diameter is in the range of from 10 to 30 nm (preferably from 10 to 25 nm) and the second filtration medium has a pore size distribution such that the modal pore diameter is in the range of from 10 to 25 nm.

According to another aspect of the invention, there is provided a method of removing particles from a feed fluid, the method comprising passing a fluid through a first filtration medium having a thickness of from 5 to 20 μm and a pore size distribution such that the modal pore diameter is in the range of from 10 to 30 nm (preferably from 10 to 25 nm); and passing the fluid through a second filtration medium having a thickness of from 20 to 70 μm (e.g. 20 to 45 μm) and a pore size distribution such that the modal pore diameter is in the range of from 10 to 25 nm; so as to retain at least a portion of the particles on each medium to produce a filtrate containing a lower concentration of the particles than the feed fluid.

With regard to particle size, the term “diameter” and “average diameter” are used interchangeably herein and mean that the average diameter of the particles is in the range of the defined limits, wherein the particles can be measured by any commonly used method, such as dynamic light scattering (DLS), transmission electronic microscopy (TEM), scattering electronic microscopy (SEM), atomic force microscopy (AFM) etc.

In the context of the present invention, the first and second filtration media, which may be, individually, cellulose based filters and, more preferably, those based on the filter paper format were found particularly useful. For avoidance of doubt and to exclude confusion, filter paper here means a filtration medium composed of cellulose fibers, which were processed into their final shape through wet-laid drying and without cellulose dissolution in suitable solvents or ionic liquids. The latter is meant to oppose those filtering media that are produced by phase inversion, herein referred to as membranes, including regenerated cellulose membranes.

The inventors have surprisingly found that passing a protein containing fluid through a series of two filter media having different thicknesses but comparable modal pore sizes, greatly enhances the throughput of the feed fluid through the second filter medium. In particular, the said first filter medium having a thickness of from 5 to 20 μm provides a particularly useful property to reject particles from above 40 nm, including soluble and insoluble particles that are not retained on 0.1-0.2 μm filters, and generally allow passage of particles below 40 nm; the second filter medium, having a thickness between 20 to 70 μm (e.g. 20 to 45 μm) provides a particularly useful property to reject particles from above 20 nm, including soluble and insoluble particles, that are not retained on the first filter paper. The rejection of 40 nm particles can be monitored by examining the intensity particle size distribution curves in the dynamic light scattering method. Additional confirmation may be found by performing gel chromatography on columns with suitable pore size. More preferably the particle rejection efficiency can be monitored by particle removal probability log10 reduction value (LRV) using monodisperse microbiological probes, such as bacteriophages. The first filter medium provides a particle removal probability log10 reduction value (LRV) of greater than or equal to 1 for particles having a diameter of below, or equal to, about 40 nm and a particle removal probability log10 reduction value (LRV) of greater than or equal to 3 for particles greater than, or equal to, about 40 nm in diameter, preferably when operated at 1 bar and up to 30 L/m² load; the second filter medium provides a particle removal probability log10 reduction value (LRV) of greater than or equal to 3 for particles having a diameter of from about 15 nm, preferably when operated at 1 bar and up to 30 L/m² load.

Preferably, the filtration media independently comprise cellulose fibres. That is to say the first and/or second filtration medium may comprise cellulose fibres.

Conveniently, the first and/or second filtration media are filter papers.

Advantageously, the cellulose fibres comprise elementary fibrils of a diameter of greater than, or equal to, about 10 nm, for example greater than about 15 nm, for example a diameter of greater than 20 nm, e.g. from 20 nm to 30 nm.

Preferably, the cellulose fibres are derived from green filamentous algae. More preferably at least half of the cellulose fibres are derived from green macroalgae, such as algae Cladophorales and/or algae Siphonocladales orders. Even more preferably, at least a portion of the cellulose fibres are derived from algae cladophora or pithophora species.

Conveniently, at least 60%, e.g. at least 70%, at least 80% or at least 90% of the cellulose fibres have a diameter of greater than 15 nm, for example a diameter greater than 20 nm, e.g. from 20 nm to 30 nm.

Preferably, at least half, e.g. at least 60%, at least 70%, at least 80% or at least 90% of the cellulose has a degree of crystallinity greater than 90%.

Advantageously, at least half of the cellulose has a degree of crystallinity of at least 95%.

Conveniently, at least 60%, e.g. at least 70%, at least 80% or at least 90% of the cellulose has a degree of crystallinity greater than 95%.

Preferably, the modal pore diameter of the first filtration medium is from 15 nm to 25 nm, such as about 16 nm, about 17 nm, about 18 nm, about 19 nm, about 20 nm, about 21 nm, about 22 nm, about 23 nm, or about 24 nm. Advantageously, less than 5% of the pore volume of the first filtration medium comprises pores of greater than about 50 nm, preferably the first filtration medium is essentially free from pores of diameter greater than about 50 nm.

Advantageously, the modal pore diameter of the second filtration medium is from 15 nm to 25 nm, such as about 16 nm, about 17 nm, about 18 nm, about 19 nm, about 20 nm, about 21 nm, about 22 nm, about 23 nm, or about 24 nm. Advantageously, less than 5% of the pore volume of the second filtration medium comprises pores of greater than about 40 nm, preferably the second filtration medium is essentially free from pores of diameter greater than about 40 nm.

Conveniently, the modal pore size difference between the two media is from about 1 to about 5 nm, such as about 3 nm.

Conveniently, the total porosity of the first and/or second filtration medium is at least 10%, such as at least 20%, for example at least greater than 30%, e.g. from 30% to 60%, preferably from 30% to 50%.

Preferably, the second filtration medium is thicker than the first filtration medium.

Advantageously, the first filtration medium has a thickness in the range of from about 5 to 19 μm, such as from about 5 to 15 μm.

Conveniently, the second filtration medium has a thickness in the range of from about 21 to 45 μm, such as from about 21 to 40 μm. In the cases when particle rejection properties are more critical than the flux, it is preferable to use second filtration medium having a thickness of about 35 μm or higher, such as from 35 μm to 45 μm, for example from 35 μm to 40 μm. Similarly, in the cases when flux is as critical as particle rejection properties, it is preferable to use second filtration medium having a thickness between 21 and 35 μm.

It has been surprisingly found that where the thickness of the second filtration medium is large, such as greater than 35 μm, especially including between 45 μm and 70 μm, the membrane is both effective and has a flux which makes it well suited for deploying in methods of continuous bioprocessing, where low flow rates are preferred.

Advantageously, the nominal upper cut-off pore diameter for the first filtration medium is about 90 nm, for example about 85 nm, about 80 nm, about 75 nm and more preferably about 70 nm based on an LRV above 4 for model large size virus (e.g. PR772 phage), when operated at 1 bar and up to 30 L/m² load.

Conveniently, the nominal upper cut-off pore diameter for the second filtration medium is about 40 nm, such as about 35 nm, about 30 nm, about 25 nm and more preferably about 20 nm based on an LRV above 4 for model small size virus (e.g. TX174 phage), when operated at least at 1 bar and up to 30 L/m² load.

Preferably, the pore size distribution of the filtration media is derived from Barett-Joiner-Halenda (BJH) N₂ gas desorption analysis (Barrett, E. P.; Joyner, L. G. Determination of Nitrogen Adsorption-Desorption Isotherms-Estimation of Total Pore Volumes of Porous Solids. Anal. Chem. 1951, 23, 791-792).

Conveniently, the log10 reduction value (LRV) is derived from the Karber-Spearman method (G. Karber, Naunyn-Schmiedebergs Arch. Exp. Pathol. Pharmakol., 1931, 162, 480-483).

Advantageously, the first and/or second filtration medium comprise a support, preferably the support comprises paper, such as paper made from plant-based cellulose. The support may, for example, comprise a filter paper of the prior art. Conveniently, the support layer and the filtration media are incorporated into a single sheet multi-layered structure.

The method may comprise the step of passing the feed fluid through at least one pre-filtration membrane prior to passing through the first filtration medium, wherein the at least one pre-filtration membrane has a pore size distribution such that the modal pore diameter is greater than, or equal to, about 100 μm, such as from about 100 μm to about 200 μm, for example greater than or equal to about 200 μm.

Conversely, the method may not comprise a pre-filtration step as detailed above.

Conveniently, the particles comprise aggregates.

The particles may be either desired particle or undesired particles. Therefore, the removal of the particles from the feed fluid may be to purify the feed fluid from undesired particles or to isolate desired particles from the feed fluid.

In either case, preferably, the method produces a filtrate containing a lower concentration of the particles than the feed fluid.

Preferably, the feed fluid comprises a product, such as a protein product, for example a plasma-derived human protein product, and the method results in greater than about 60% yield of the product after filtration through the two media, such as greater than about 65%, 70%, 75%, 80%, 85%, 90%, or 95% yield after filtration through the two media. More preferably, the method results in greater than about 96%, 97%, 98% or 99% yield after filtration through the two media.

Conveniently, the particles have an average diameter of greater than, or equal to, about 10 nm, for example an average diameter of greater than about 12 nm, about 15 nm, about 18 nm, about 20 nm, about 30 nm, about 40 nm, about 100 nm, or about 200 nm.

Preferably, the particles have an average diameter of from about 10 nm to about 200 nm, such as about 10 nm to about 100 nm.

Conveniently, the particles have a bi- or multimodal distribution with a first average diameter of from about 18 nm to about 50 nm, for example from about 20 nm to about 40, and a second average diameter of from about 50 nm to about 90 nm, such as from about 60 nm to about 80 nm.

Advantageously, the particles comprise proteins such as soluble and insoluble protein aggregates, high molecular weight protein impurities (200 kDa), unfolded or misfolded proteins, and other undesired impurities, e.g. cell debris, nucleic acids, etc.

Preferably, the soluble protein aggregates have an average diameter of greater than about 40 nm, for example from about 50 nm to about 90 nm, such as from about 60 nm to about 80 nm.

Advantageously, the undesired particles comprise microorganisms, such as viruses. Furthermore, the undesired particles may comprise proteins such as prion (protein) particles (PrPs). For example, undesired virus particles typically have an average diameter greater than about 18 nm, such as from about 18 nm to about 50 nm, for example from about 20 nm to about 40. Whereas, undesired prion particles typically have an average diameter of about 12 nm.

Preferably, the particles comprise a mixture of protein aggregates, unfolded or misfolded proteins, PrPs, and/or viruses as well as other undesired impurities, e.g. cell debris, nucleic acids, etc.

Where the particles comprise viruses, it is preferred that the overall method provides a virus removal probability log10 reduction value (LRV) of greater than, or equal to, 4 e.g. greater than, or equal to, 5 or 6 for particles having a diameter of from about 10 to about 40 nm.

Advantageously, when passing the feed fluid through the first filtration medium this provides a particle removal probability log10 reduction value (LRV) of greater than, or equal to 1, for example greater than, or equal to, 2, 3, or 4, such as from 1 to 4, or 1 to 3, or 1 to 2, for particles having a diameter of from about 10 to about 40 nm.

Conveniently, when passing the feed fluid through the first filtration medium this provides a particle removal probability log10 reduction value (LRV) of greater than, or equal to, 3 or 4 for particles greater than, or equal to, about 40 nm in diameter, such as an LRV of greater than, or equal to, 5 for particles greater than, or equal to, about 40 nm.

Preferably, when passing the feed fluid through the first filtration medium this provides a particle removal probability log10 reduction value (LRV) of greater than, or equal to, 3 or 4, such as an LRV of greater than, or equal to, 5 for particles greater than, or equal to, about 50 nm, 60 nm, or 70 nm, such as particles having a diameter of from 50 nm to 100 nm, 50 nm to 90 nm, and/or 50 nm to 80 nm.

Preferably, when passing the feed fluid through the second filtration medium this provides a particle removal probability log10 reduction value (LRV) of greater than, or equal to, 3 or 4 for particles having a diameter of from about 10 to about 40 nm, such as an LRV of greater than, or equal to, 5 for particles from about 10 to about 40 nm in diameter.

Advantageously, when passing the feed fluid through the second filtration medium this provides a particle removal probability log10 reduction value (LRV) of greater than, or equal to, 3 or 4 for particles having a diameter of greater than, or equal to, about 40 nm, such as an LRV of greater than, or equal to, 5 for particles greater than, or equal to, about 40 nm in diameter.

Conveniently, the method as a whole provides a particle removal probability log10 reduction value (LRV) of greater than, or equal to, 1, e.g. greater than, or equal to, 2, 3, 4 or 5 for particles greater than, or equal to, 10 nm in diameter; and/or the method provides a LRV greater than, or equal to, 3, 4, or 5 for particles greater than, or equal to, 18 nm or 20 nm in diameter; and/or the method provides an LRV greater than, or equal to, 5 or 6 for particles greater than, or equal to, 40 nm in diameter. For small viruses it is preferred that the LRV is greater than, or equal to, 3. For large viruses it is preferable that the LRV is greater than, or equal to, 4. For prion particles it is preferred that the LRV removed is greater than, or equal to, 1 and more preferably greater than, or equal to, 3.

Advantageously, the feed fluid is passed through the filtration media under a pressure differential of approximately 3 to 600 kPa, so that the integrity of the filter media is not compromised. The pressure differential may be applied either as overhead pressure (for example an overhead pressure of 3 to 600 kPa, e.g. 100 to 300 kPa) or as suction pressure (for example at a suction pressure of 3 to 600 kPa).

Conveniently, the method of the invention can be used in the purification of plasma-derived human proteins, for example, but not limited to, human serum albumin (HSA).

In certain embodiments, the first and second media are independently interchangeable.

According to the further aspect of the invention, there is provided a kit-of-parts comprising a first filtration medium having a thickness of from 5 to 20 μm, wherein the first filtration medium provides a particle removal probability log10 reduction value (LRV) of greater than or equal to 1 for particles having a diameter of from about 10 to about 40 nm and a particle removal probability log10 reduction value (LRV) of greater than or equal to 3 for particles greater than about 40 nm in diameter; and

a second filtration medium providing a particle removal probability log10 reduction value (LRV) of greater than or equal to 3 for particles having a diameter of from about 10 to about 40 nm and a particle removal probability log10 reduction value (LRV) of greater than 3 for particles having a diameter of greater than about 40 nm.

Preferably, the second filtration medium has a thickness of from 20 to 70 μm (e.g. 20 to 45 μm).

Preferably, the first filtration medium has a pore size distribution such that the modal pore diameter is in the range of from 10 to 30 nm (preferably from 10 to 25 nm) and wherein the second filtration medium has a pore size distribution such that the modal pore diameter is in the range of from 10 to 25 nm.

In another aspect of the invention, there is provided a first filtration medium having a thickness of from 5 to 20 μm and a pore size distribution such that the modal pore diameter is between 10 and 25 nm; and a second filtration medium having a thickness of from 20 to 45 μm and a pore size distribution such that the modal pore diameter is between 10 and 25 nm.

Preferably, the filtration media of the kit-of-parts comprise cellulose fibres, wherein the cellulose fibres may have any of the characteristics of the cellulose fibres as described above in relation to the first aspect of the invention.

Advantageously, the first and second media of the kit-of-parts are independently interchangeable.

Any of the embodiments outlined above in relation to the method can be attributed to the embodiments directed towards the kit-of-parts where applicable.

According to another aspect of the invention, there is provided the use of a kit-of-parts as described above for removing particles from a feed fluid. Preferably, the kit-of-parts is used for the purification of plasma-derived human proteins, for example human serum albumin (HSA).

The invention provides a method for the removal of undesired protein aggregates and/or high molecular weight impurities from monomeric protein solutions, resulting in increased product yield, stable flux and high virus removal efficiency. Preferably, the method involves a two-step process of removing aggregates using nanocellulose-based filter media. Herein, the first filter medium is designed to remove the high molecular weight impurities from monomeric protein solutions, whereas the second filtration medium allows for high product yield.

This method prevents rapid filter fouling of the finer second filtration medium beyond what is expected. In more detail, when using a commonly used prefilter with a pore size of 0.1 to 0.2 μm, the finer medium for virus removal still experiences rapid fouling.

However, the inventors have surprisingly found that the use of a first filtration medium, as outlined above, extends the lifetime of the second filtration medium much more than expected. This is surprising as the modal pore size of the first filter medium is not sufficiently different to reject passage of relatively large particles through. Therefore, based on the modal pore size distributions, the ability of the first filter medium to efficiently purify the feed solution from undesired contaminants is totally unexpected. However, as detailed below, using the first filter medium in series extends the lifetime of the second filter medium significantly beyond what was to be expected and limited fouling of the second filter medium was evidenced across the load volumes analysed.

This method has, therefore, a number of advantages which include:

1. increased product recovery due to the reduction in reliance on commonly used pre-filters which are known to lead to product loss due to non-specific adsorption of media components;

2. faster processing times of feed fluids; and

3. reduced costs due to the extended lifetime of the finer second filtration medium.

4. reduced quantities of potentially immunogenic impurities

Embodiments of the present invention may be useful in relation to separation of soluble and insoluble protein aggregates, high molecular weight impurities, unfolded or misfolded proteins, and other contaminants from monomeric proteins based on the size exclusion principle; removal of viruses from small-molecule size active pharmaceutical ingredients, including in relation to ophthalmic uses; removal of nanoparticles for medical use containing genetic material, which are sensitive to sterilization, including not only biological systems, e.g. viruses, but also artificially made gene delivery systems, e.g. polymeric nanoparticles, solid lipid nanoparticles and/or liposomes.

### DESCRIPTION OF THE FIGURES

Embodiments of the present invention will now be described with reference to the following drawings:

FIG. 1 is a graph showing filter flux using a 10 g/L HSA solution in a two-step filtration process with 11 μm and 22 μm nanocellulose based filters at 1 bar.

FIG. 2 is a graph showing the comparison of second step filtration with 33 μm nanocellulose based filters at 1 bar and 3 bar.

FIG. 3 is a graph showing the dynamic light scattering (DLS) analysis of HSA solution representing feed, pre-filtrate 11 μm, and filtrate 22 μm at 1 bar.

FIG. 4 is three graphs, showing the results of size exclusion gel chromatography (ÄKTA) of 10 mg/ml HSA feed, pre-filtrate 11 μm, and filtrate 22 μm at 1 bar.

FIG. 5 is a graph showing the LRV of ΦX174 phage-spiked 10 mg/ml HSA filtrate with 22 μm filter at 3 bar.

FIG. 6 is a graph showing filter flux using a 50 g/L HAS solution in a two-step filtration process with 11 μm and 22 μm nanocellulose based filters at 1 bar.

### EXAMPLES

The present invention will be further described by reference to the following non-limiting examples.

### Example 1

The invention can be illustrated by one of the most common plasma-derived human proteins, i.e. human serum albumin (HSA). HSA has many functions in the body, among which binding and transport properties of various hydrophobic substances is among most important functions. HSA is also an important supplement for cell culture media especially in cell therapies. It can be used for cryopreservation of cell therapies. Therefore, it is critical that human serum albumin is virus- and other pathogen free for these applications. Albumin is a single polypeptide chain of 585 amino acids with molecular weight around 65-67 kDa. The structure is tightly coiled due to numerous thiolic bonds. It is well established that HSA may undergo extensive aggregation due to disulfhydryl binding and various hydrophobic interactions. For this reason, HSA is a suitable model protein to demonstrate the invention.

A commercially available HSA sample (200 mg/ml) was purchased from a local pharmacy store. The sample was diluted with phosphate buffer solution (PBS) pH 7.4 to 10 g/L concentration.

The cladophora cellulose dispersion was prepared by passing the starting cellulose material through a high-pressure microfluidizer (Microfluidics, MA, USA; LM20) to disperse the cellulose fibre bundles into individual nanofibres. The dispersion was passed 3 times through a 200 μm grid chamber and 1 time through a 100 μm grid chamber under a pressure of 1800 bar.

The filters were prepared as previously described (Manukyan et al., J Mem Sci, Vol. 572, 2019, 464-474). The diluted dispersion was drained through a medium (Durapore; 0.65 μm DVPP; Merck Millipore, MA, USA) using a vacuum filtration setup (Advantec, Japan) until a cellulose cake was formed on top of the medium. The wet cake was then removed and dried at the desired temperature and time depending on the type of filter using a hot press (Carver, IN, USA; 4122CE). For the preparation of 11 μm thick pre-filters, 50 ml of 1 mg/ml nanocellulose suspension was used, and the nanocellulose filtrate cake was dried at 140° C. using a hot-press for 40 min. For the preparation of 22 μm thick virus removal filter, 100 ml of 1 mg/mL of nanocellulose suspension was used, and the nanocellulose filtrate cake was dried at 80° C. using a hot-press for 24 h. The dry filters were removed, cut into 47 mm diameter discs.

Filtration Setup

An Advantec KST-47 (Japan) filter holder was used. A general purpose filter paper disc (47 mm in diameter, Munktell) was placed beneath the nanocellulose filter as support. The rate of flow was monitored gravimetrically by collecting the outflowing liquid on an analytic balance (Mettler Toledo, Switzerland), connected to LabX software (Version 2.5, Mettler Toledo, Switzerland) at 20 second intervals.

Pre-Filtration

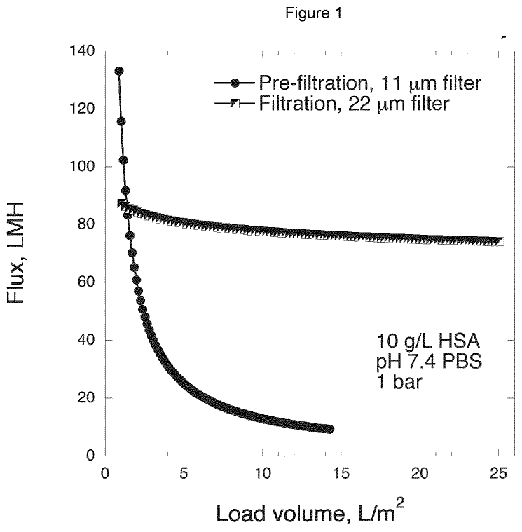

11 μm filters were used in a first filtration step. The removal was validated on single sheet filters. Feed solution was 10 g/L HSA diluted in PBS and adjusted to pH 7.4. The filters were wetted with PBS prior to filtration. Filtrations through the 11 μm filters were carried out at 1 bar. Due to rapid fouling, for each filtration through the 11 μm filters around 25 mL was passed through each 11 μm filter corresponding to 14-15 L/m² load volume (FIG. 1). The solution was then passed through a 22 μm filter and little fouling was observed. The permeate fractions were collected, mixed together, and stored at 4° C. before usage.

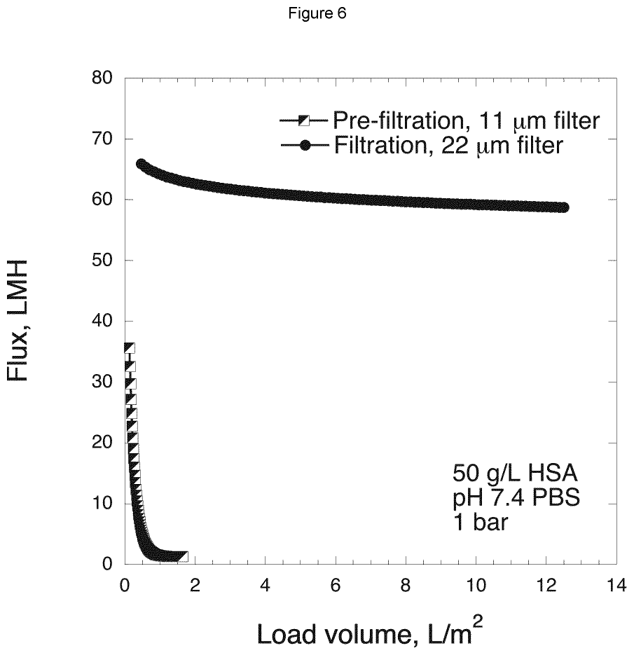

The same procedure was carried out for a feed solution of 50 g/L HAS. Around 10 ml was passed through each 11 μm filter corresponding to about 7 L/m² load volume. The solution was then passed through a 22 μm filter and little fouling was observed (FIG. 6).

Filter Flux and Fouling Behaviour

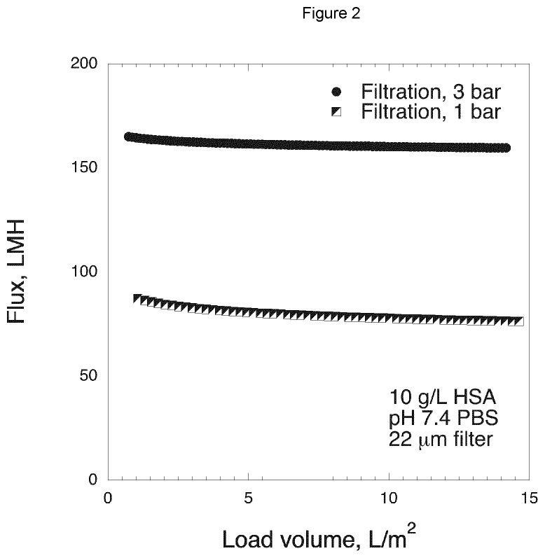

FIGS. 1, 2, and 6 show the flux and fouling behaviour of different filters at different pressures or different protein concentrations. FIG. 1 shows that the 11 μm filter rapidly fouls when HSA 10 g/L solution is passed through it at 1 bar. Second filtration at 1 bar does not result in filter fouling and the flux is steady. FIG. 2 shows that the 22 μm filter does not foul even when it is operated at 3 bar, using an 11 μm pre-filtered solution. No fouling was observed at both pressures. FIG. 6 shows that when the concentration of HSA is increased to 50 g/L the fouling is even more rapid in the 11 μm filter. However, second filtration of the same solution through the 22 μm filter does not result in filter fouling, and the flux is stable throughout the experiment. The protein recovery after second filtration was high, indicating that only minor protein loss during two-step filtration.

For a 1% solution of HSA, 87% of product was obtained after filtering through the 11 μm thick filter and 85% of product was recovered through the following step of filtration through the 22 μm. For a 5% solution of HSA the recoveries were 94 and 93% respectively.

Monitoring of Particle Size Distribution and Removal of Aggregates

Dynamic Light Scattering

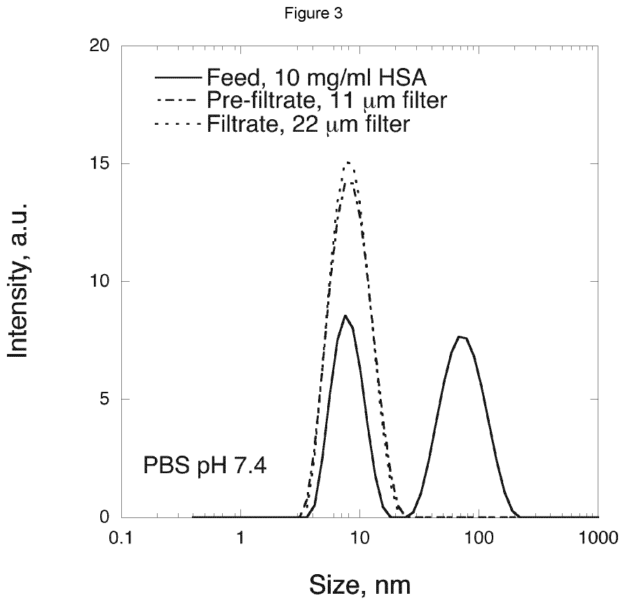

Dynamic light scattering (DLS) was used to assess particle size distribution of 10 g/L HSA solution in PBS pH 7.4 with Nano ZS instrument (Malvern, UK), see FIG. 3.

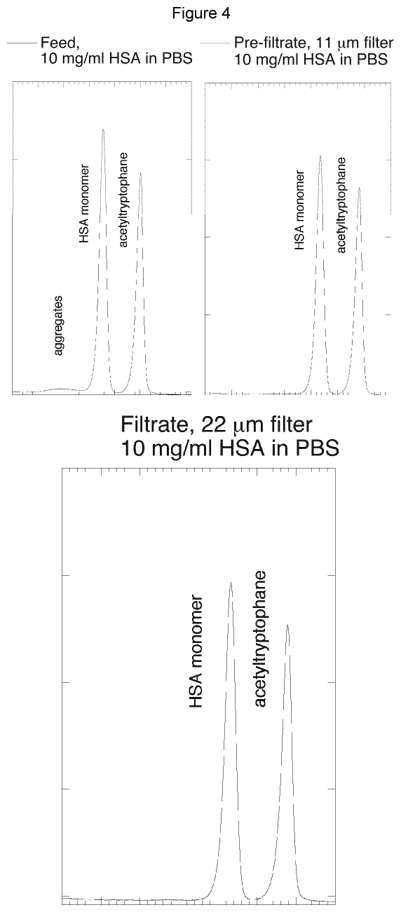

SEC-ÄKTA chromatography Protein Purification of HSA-PBS solution using Size Exclusion Chromatography (ÄKTA START) instrument. Selected chromatographic column (Mw 40-20,000 kDa; HiPrep 26/60 Sephacryl S-500HR, GE, Uppsala, Sweden) was used with a flow velocity of 1 ml/min. The column was equilibrated with 0.5 cV of PBS buffer and then 3.2 ml 1 wt. %. The purified solution was collected using peak fractionating in 10 ml falcon tubes and the fractions were collected when absorbance was ≤5 mAU, see FIG. 4.

To explain the observed enhanced throughput capacity in the second filtration step as compared to the first one, DLS and ÄKTA chromatography were performed. FIG. 3 shows the results of intensity particle size distributions for samples presented in FIG. 1. It is seen that the second peak in the feed solution, that is stretching in the region between 30 and 200 nm, is not detectable after filtration through 11 μm and 22 μm filters.

Size exclusion gel chromatography with ÄKTA confirmed the removal of trace quantities of large molecular weight fractions from the feed solution following filtration through 11 μm filter.

Virus Removal Filtration

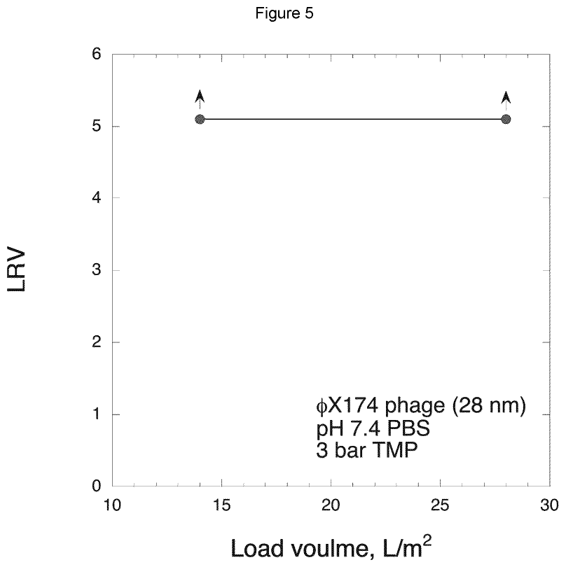

To further illustrate the invention and to confirm that 22 μm filter retains high virus removal capacity after prefiltration, PFU tests were performed. FIG. 5 shows the results of ΦX174 bacteriophage (28 nm) from 10 g/L HSA solution.

22 μm and 11 μm filters were used for virus removal studies using single sheet filters. Prior to virus removal filtration, the virus-spiked feed solution was pre-filtered through a 0.2 μm filter (VWR). A highly purified ΦX174 stock solution was spiked at 0.1% into the pre-filtrated 10 mg/ml HSA in PBS, adjusted to specific pH. Virus stability was controlled by a hold sample taken from the pre-filtered spiked feed solution. The filters were wetted with PBS prior to filtration.

The PFU assay was used as previously described (Manukyan et al., J Mem Sci, Vol. 572, 2019, 464-474). Escherichia coli bacteriophage ΦX174 (ATCC 13706-B1 ™) Escherichia coli bacteriophage PR772 (ATCC® BAA769B1 ™) and the host bacteria Escherichia coli (Migula) Castellani and Chalmers (E. coli) (ATCC 13706) were obtained from ATCC (Manassas, Va., USA). The titer of bacteriophages was determined by plaque forming units (PFU) assay. The feed and permeate samples were serially diluted in Luria-Bertani medium (LBM) (1% tryptone, 0.5% yeast extract, and 1% NaCl in deionized water), and 100 μl of diluted bacteriophage was mixed with 200 μl of E. coli stock. The resulting suspension was mixed with 1 ml of melted soft agar and poured on the surface of prepared hard agar plate (55×15 mm) and incubated at 37° C. for 5 hours. The feed titer was adjusted to about 10⁵ to 10⁶ bacteriophages ml⁻¹. The limit of detection, i.e. 0.7 PFU ml⁻¹, of the current experimental design refers to 5 bacteriophages ml⁻¹, corresponding to a single detectable plaque in one of the plates for non-diluted duplicate samples, assuming that at the detection limit each plaque is produced by one bacteriophage. The virus retention was expressed as logo reduction value (LRV).

### Example 2

Nominal Pore Size of Filters

ΦX174 bacteriophage (28 nm) and PR772 phage (70 nm) were used as a model monodispersed probes and virus removal quantification using 11 and 33 μm filters. The tests were carried at 1 bar pressure using 50 mL of PBS corresponding to 26 L/m² load volume. Permeate samples and hold samples were collected and stored at 4° C. before PFU assay. FIG. 5 shows the LRV value obtained after filtering through the second (33 μm) filter.

Plaque Forming Units (PFU) and Log10 Reduction Value (LRV) The PFU assay was used as previously described (Manukyan et al., J Mem Sci, Vol. 572, 2019, 464-474). The titer of ΦX174 bacteriophage was determined by plaque forming units (PFU) assay. The feed and permeate samples were serially diluted in Luria-Bertani medium (LBM) (1% tryptone, 0.5% yeast extract, and 1% NaCl in deionized water), and 100 μl of diluted bacteriophage was mixed with 200 μl of E. coli stock. The resulting suspension was mixed with 1 ml of melted soft agar and poured on the surface of prepared hard agar plate (55×15 mm) and incubated at 37° C. for 5 hours. The feed titer was adjusted to about 10⁵ to 10⁶ bacteriophages ml⁻¹. The limit of detection, i.e. 0.7 PFU ml⁻¹, of the current experimental design refers to 5 bacteriophages ml⁻¹, corresponding to a single detectable plaque in one of the plates for non-diluted duplicate samples, assuming that at the detection limit each plaque is produced by one bacteriophage. The virus retention was expressed as logo reduction value (LRV).

1. A method of removing particles from a feed fluid, the method comprising:
passing the fluid through a first filtration medium having a thickness of from 5 to 20 μm, wherein passing the feed fluid through the first filtration medium provides a particle removal probability log10 reduction value (LRV) of greater than or equal to 1 for particles having a diameter of from about 10 to about 40 nm and a particle removal probability log10 reduction value (LRV) of greater than or equal to 3 for particles greater than about 40 nm in diameter; and
passing the fluid through a second filtration medium having a thickness of from 20 to 70 μm (e.g. 20 to 45 μm), wherein passing the feed fluid through the second filtration medium provides a particle removal probability log10 reduction value (LRV) of greater than or equal to 3 for particles having a diameter of from about 10 to about 40 nm and a particle removal probability log10 reduction value (LRV) of greater than or equal to 3 for particles having a diameter of greater than or equal to about 40 nm; so as to retain at least a portion of the particles on each medium to produce a filtrate containing a lower concentration of the particles than the feed fluid.

2. The method according to claim 1, wherein the first filtration medium has a pore size distribution such that the modal pore diameter is in the range of from 10 to 30 nm (preferably 10 to 25 nm) and wherein the second filtration medium has a pore size distribution such that the modal pore diameter is in the range of from 10 and 25 nm.

3. The method according to claim 1, wherein the filtration media comprise cellulose fibres.

4. The method according to claim 3, wherein the cellulose fibres comprise elementary fibrils of a diameter of greater than or equal to about 10 nm.

5. The method according to claim 1, wherein the particles are selected from aggregates, high molecular weight protein impurities, unfolded or misfolded proteins.

6. The method according to claim 1, wherein the particles are selected from proteins such as soluble and insoluble protein aggregates, high molecular weight protein impurities, unfolded or misfolded proteins, and/or protein prion particles.

7. The method according to claim 1, wherein die particles comprise microorganisms, such as viruses.

8. The method according to claim 1, wherein the particles have a diameter of greater than, or equal to, about 10 nm.

9. The method according to claim 1 wherein passing the teed fluid through the first filtration medium provides a particle removal probability log10 reduction value (JRV) of greater than or equal to 2, for particles having a diameter of from about 10 to about 40 nm.

10. The method according to claim 1 wherein passing the feed fluid through the first filtration medium provides a particles removal pro liability log10 reduction value (LRV) of greater than or equal to 4 for particles greater than, or equal to, about 40 nm in diameter.

11. The method according to claim 1 wherein passing the feed fluid through the second filtration medium provides a particle removal probability log10 reduction value (LRV) of greater than or equal to 4 for particles having a diameter of from about 10 to about 40 nm.

12. The method according to claim 1, wherein passing the feed fluid through the second filtration medium provides a particle removal probability log10 reduction value (LRV) of greater than or equal to 4 for parricles having a diameter of greater than, or equal to, about 40 nm.

13. The method according to claim 1, wherein the fluid is passed through the filtration media under a pressure differential of approximately 3 to 600 kPa.

14. The method according to claim 1, wherein the first and second medium are independently interchangeable.

15. The method according to claim 1, wherein the method comprises the step of passing the feed fluid through at least one pre-filtration membrane prior to passing through the first filtration medium, wherein the at least one pre-filtration membrane has a pore size distribution such that the modal pore diameter is greater than, or equal to, about 100 μm.

16. A kit-of-parts comprising:
a first filtration medium having a thickness of from 5 to 20 μm, wherein the first filtration medium provides a particle removal probability log10 reduction value (LRV) of greater than or equal to 1 for particles having a diameter of from about 10 to about 40 nm and a particle removal probability log10 reduction value (LRV) of greater than or equal to 3 for particles greater than or equal to about 40 nm in diameter; and
a second filtration medium having a thickness of from 20 to 70 μm (e.g. 20 to 45 μm), wherein the second filtration medium provides a particle removal probability log10 reduction value (LRV) of greater than or equal to 3 for particles having a diameter of from about 10 to about 40 nm and a particle removal probability log10 reduction value (LRV) of greater than or equal to 3 for particles having a diameter of greater than about 40 nm.

17. The kit-of-parts according to claim 16, wherein the first filtration medium has a pore size distribution such that the modal pore diameter is in the range of from 10 to 30 nm (preferably troth 10 to 25 nm) and wherein the second filtration medium has a pore sire distribution such that the modal pore diameter is in the range or from 10 to 25 nm.

18. The kit-of-parts according to claim 16, wherein the filtration media comprise cellulose fibres.

19. The kit-of-parts according to claim 18, wherein the cellulose fibres comprise elementary fibrils of a diameter of greater than about 10 nm.

20. The kit-of-parts according to claim 16, wherein the first and second media of the kit are independently interchangeable.

21. The use of a kit-of-parts according to claim 16 for removing particles from a feed fluid.
